(19) United States
(12) Patent Application Publication
(10) Pub. No.: US 2024/0164687 A1
(43) Pub. Date: May 23, 2024

(54) System and Method for Blood Pressure Assessment

(71) Applicant: ChroniSense Medical Ltd., Yokneam Illit (IL)

(72) Inventors: Assaf Pressman, Pardes Hanna-Karkur (IL); Din Hadass, Ramat Yishai (IL); Vladimir Muzykovski, Yokneam Illit (IL); Daniel H. Lange, Kfar Vradim (IL)

(21) Appl. No.: 18/427,229

(22) Filed: Jan. 30, 2024

Related U.S. Application Data
(63) Continuation-in-part of application No. 18/127,514, filed on Mar. 28, 2023, now Pat. No. 11,931,155.
Application No. 18/127,514 is a continuation of application No. 14/738,636, filed on Jun. 12, 2015, now Pat. No. 11,712,190.

(51) Int. Cl. A61B 5/318; A61B 5/00; A61B 5/0205; A61B 5/024; A61B 5/0245; A61B 5/103; A61B 5/11; A61B 5/30; A61B 5/316; G16H 40/63; G16H 80/00

(52) U.S. Cl. CPC A61B 5/318; A61B 5/0205; A61B 5/02416; A61B 5/02427; A61B 5/02438; A61B 5/0245; A61B 5/1032; A61B 5/30; A61B 5/316; A61B 5/4842; A61B 5/681; A61B 5/6822; A61B 5/6824; A61B 5/6829; A61B 5/721; A61B 5/7239; A61B 5/7275; G16H 40/63; G16H 80/00; A61B 5/0022; A61B 5/11; A61B 2562/046; A61B 2562/06

(57) ABSTRACT

Provided are systems and methods for blood pressure assessment using a wrist and cardiac device. In one embodiment, the wrist device receives a synchronization pulse, indicating cardiac onset, and generates blood-arrival signal data. In another embodiment, the wrist device receives cardiac signal data and generates blood-arrival signal data. The synchronization pulse or the cardiac signal data and the blood-arrival data are synchronized. The synchronization pulse or cardiac signal data and the blood-arrival signal data are processed to determine a pulse transit time between the heart and the wrist device. This pulse transit time and patient parameter are input into a trained neural network to generate an assessed blood pressure. The cardiac signal data can be generated by an electrical, acoustical, echocardiographic, or ballistocardiograph sensor. The blood-arrival signal data can be generated by an optical sensor, a tonometry sensor or a pressure-sensing sensor.

### CROSS-REFERENCE TO RELATED APPLICATIONS

The present application is a continuation-in-part of and claims priority benefit of U.S. patent application Ser. No. 18/127,514, filed Mar. 28, 2023. The U.S. patent application Ser. No. 18/127,514 is a continuation of U.S. patent application Ser. No. 14/738,636, filed Jun. 12, 2015, now U.S. Pat. No. 11,712,190, issued on Aug. 1, 2023, entitled “Wearable Device Electrocardiogram.” The present application is also related to U.S. patent application Ser. No. 14/738,666 titled “Monitoring Health Status of People Suffering from Chronic Diseases” filed on Jun. 12, 2015, now U.S. Pat. No. 11,160,459, issued on Nov. 2, 2021. The present application is further related to U.S. patent application Ser. No. 14/738,711 filed on Jun. 12, 2015, now U.S. Pat. No. 10,470,692, issued on Nov. 12, 2019, entitled “System for Performing Pulse Oximetry”. The subject matter of the aforementioned applications is incorporated herein by reference for all purposes.

### FIELD

The present application relates to systems and methods for blood pressure assessment, and more specifically, using a trained machine learning system or a regression fitted function to provide a blood pressure assessment based on pulse transit time and other patient parameters.

### BACKGROUND

Various aspects discussed herein are directed to devices for blood pressure measurement.

There are problems with current non-invasive blood pressure measurement devices.

Accordingly, there is a need in the art for improved blood pressure measuring devices.

### SUMMARY

This summary is provided to introduce a selection of concepts in a simplified form that are further described below in the Detailed Description. This summary is not intended to identify key features or essential features of the claimed subject matter, nor is it intended to be used as an aid in determining the scope of the claimed subject matter.

According to one aspect of the present disclosure, a system is disclosed for blood pressure assessment. The blood pressure assessment process can be repeated to provide a continuous blood pressure monitoring system for a patient. A calculated PTT (pulse transit time) and patient parameters are processed by a trained neural network or a function that has previously been curve-fitted using a regression process. In some embodiments, a cardiac device, a wrist device, and a synchronization device are used. In another embodiment, only a cardiac device and a wrist device is used. In a further embodiment, the blood pressure assessment only requires a wrist device for the blood pressure assessment.

The cardiac device and wearable device can include a means to be synchronized so that the timing between the cardiac signal data and the blood-arrival signal data can be determined by utilizing the synchronization signal. The synchronization means can be internal or external to the devices. In one embodiment, a global synchronization source is used. This can include using GPS (Global Positioning System) signals or cellular system signals. A local external synchronization component can include a low-power device that transmits a timing clock.

In the further embodiment, the cardiac sensor and the blood-arrival sensor are both located in the wrist device along with the synchronization means.

Synchronization of the devices can be implemented by sending a start message or time-tagging the data from the sensors. A processor can be used to align the data and determine a PTT.

In another embodiment, synchronization is provided by the cardiac device sending a wired or wireless pulse from the cardiac device and reception by the wrist device. The synchronization pulse preferably has a low transmission delay or a deterministic delay between the cardiac device and the wrist device. The generation of the synchronization pulse timing corresponds with the cardiac onset and is indicated with the rising edge of the “R” phase or “R” peak of the “qRs” complex.

The sensors that can generate cardiac signal data can include an electrical sensor, an acoustical sensor, an echocardiographic sensor, and a ballistocardiograph sensor. The sensor that generates the blood-arrival signal data can include one or more optical sensors, a tonometry sensor, and a pressure-sensing sensor.

A preconfigured function is used to relate the patient's PTT and parameters to the patient's blood pressure assessment. This is referred to as the assessed blood pressure. This relation can be performed by a trained neural network or a function where regression has been used to relate the PTT parameters to an assessed blood pressure. The neural network needs to be trained using sufficient patient data having a distribution of one or more parameters of gender, age, weight, body mass index, health status, and other patient parameters.

Other example embodiments of the disclosure and aspects will become apparent from the following description taken in conjunction with the following drawings.

### BRIEF DESCRIPTION OF THE DRAWINGS

Embodiments are illustrated by way of example and are not limited to the figures of the accompanying drawings, in which like references indicate similar elements.

FIG. 1 is a block diagram showing an example system for measuring ECG data using a wearable device.

FIG. 2 is a schematic diagram showing components of an example device for measuring ECG data using the wearable device.

FIG. 3A is a schematic diagram illustrating an example device for measuring ECG data using the wearable device.

FIG. 3B is a block diagram showing an example of an optical sensor.

FIG. 4 shows example plots of noisy ECG data, a “clean” ECG waveform, and a PPG derivative.

FIG. 5 is a flow chart showing an example method for measuring ECG data using the wearable device.

FIG. 6 shows example plots of a raw PPG signal, a filtered PPG signal, an electrical signal from the left wrist, average ECG data from the left wrist, and average differential ECG data.

FIG. 7 shows example plots of time-correlated ECG data and a blood-arrival signal showing the PTT (pulse arrival time).

FIG. 8A is a block diagram showing an example system or blood pressure assessment with an external synchronization source, a cardiac device, and a wrist device.

FIG. 8B is a block diagram showing an example system or blood pressure assessment with a cardiac device, a wrist device, and the synchronization source located within the wrist device.

FIG. 8C is a block diagram showing an example system or blood pressure assessment with a wrist device and the synchronization source located within the wrist device.

FIG. 9 is a schematic diagram illustrating an example device for measuring ECG data, blood-arrival, and data using the wearable device.

FIG. 10 is a schematic diagram showing components of an example device for generating blood pressure assessment using a wrist and cardiac device.

FIG. 11 is a flow chart showing an example method for continuous blood pressure assessment using a wrist and cardiac device, synchronized by a wireless pulse associated with the “R” heart phase.

FIG. 12 is a flow chart showing an example method for measuring blood pressure assessment using the wrist and cardiac device.

FIG. 13 is a block diagram of a neural network that can be trained to generate a blood pressure assessment from a PTT and the patient parameter input.

### DETAILED DESCRIPTION

The following detailed description includes references to the accompanying drawings, which form a part of the detailed description. The drawings show illustrations in accordance with exemplary embodiments. These exemplary embodiments, which are also referred to herein as “examples,” are described in enough detail to enable those skilled in the art to practice the present subject matter. The embodiments can be combined, other embodiments can be utilized, or structural, logical and electrical changes can be made without departing from the scope of what is claimed. The following detailed description is, therefore, not to be taken in a limiting sense, and the scope is defined by the appended claims and their equivalents.

### Blood Pressure Assessment Systems and Methods

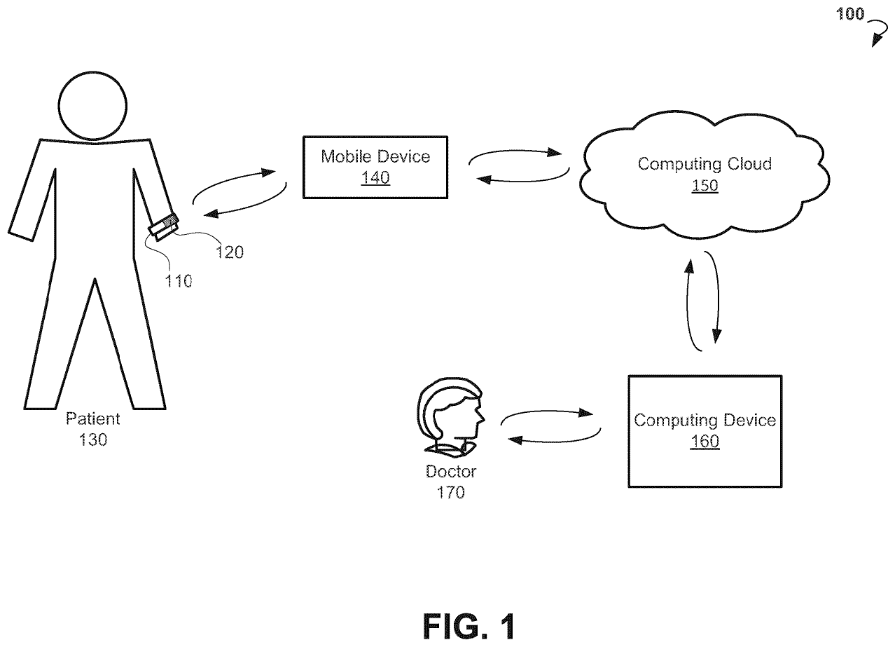

The present disclosure, notwithstanding FIGS. 1-6, provides systems and methods for continuous non-invasive blood pressure assessment of a patient using Pulse Transit Time (PTT) and patient parameters. These embodiments are suitable for multiple patient environments including, home, work, outdoors, or while the patient is located in some other stationary or mobile environment. The time delay between the cardiac onset and the blood-arrival time into a proximal part of the limb is measured. Cardiac onset corresponds to when the heart contraction occurs. Other patient parameters are input either into a trained neural network or into a multi-variable function where the parameter coefficients have been fitted through regression to fit prior baseline verified blood pressure data with PTT times and verified by a reliable means of blood pressure measurement.

Cardiac onset detection can be provided by detecting physiological attributes and signals. The systems and devices for the detection of cardiac onset can include, but are not limited to, the detection and processing of the electrocardiogram (ECG) signal, measuring impedance changes, detection of acoustical signals, and ballistocardiograph signals.

The measurement of blood-arrival time into a proximal part of the limb can be detected by using a photoplethysmography signal (PPG) using one or more optical sensors, as described above. Other methods of measuring the blood-arrival time can include, but are not limited to, tonometry methods and pressure/force measuring methods.

An important aspect of various system embodiments is the synchronization of the cardiac onset time and the blood-arrival time. The signals correlated with the cardiac onset and blood-arrival time, in some embodiments, are generated from sensor components at different physical locations on a patient. Processing steps include sampling, processing for event detection, and transmission, which can utilize different clocks. The relative time between an event detected or recorded on one physical device and another physical device needs to be synchronized. Thus, to accurately determine the PTT (cardiac onset time and blood-arrival time), time synchronization between the devices data is required.

Additionally, the clock speed on each physical device can be different. Thus, the physical device components that determine the PPT need to know the time interval between each sample to process the data accurately and determine the PPT. This synchronization can be performed by time synchronization of the differing physical devices or time-tagging data shared between the devices.

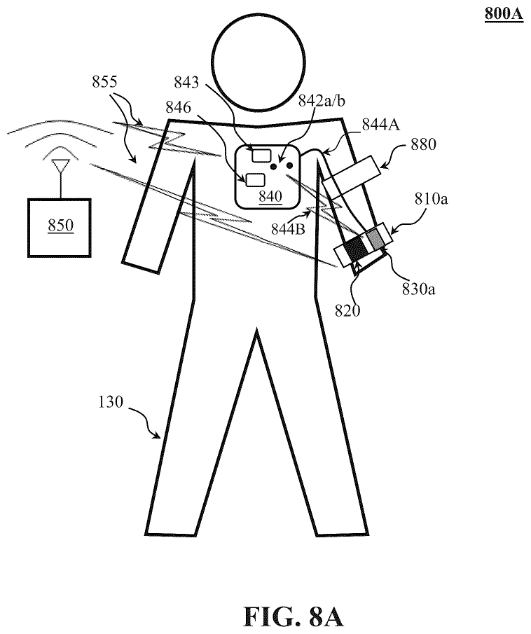

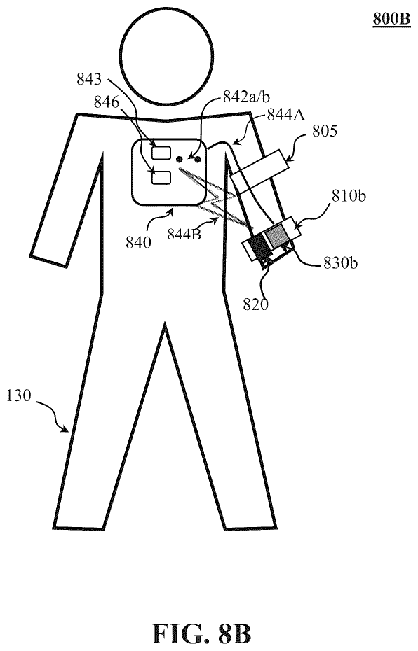

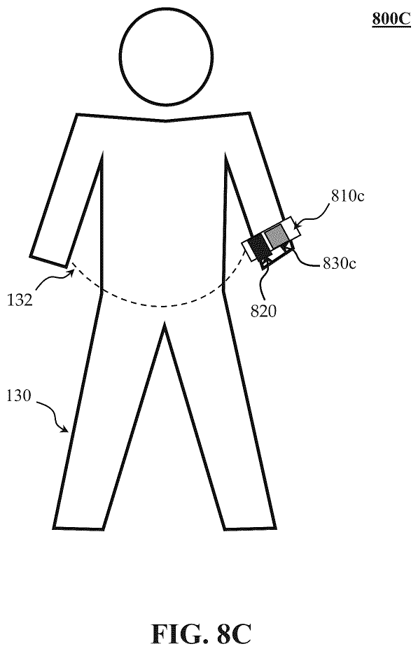

FIGS. 8A and 8B, described in detail below, depict a system utilizing a centralized electronic element for broadcasting a baseline time for synchronization. In FIG. 8A, a separate device is utilized to broadcast the synchronization signal. FIG. 8B depicts a system where synchronization is provided through communications sent between the cardiac and wrist devices. In one embodiment, a synchronization pulse is sent by the cardiac device to the wrist device. In this embodiment, the cardiac signal data does not have to be sent to the wrist but requires a low-delay or substantially deterministic wired or wireless pulse that indicates the cardiac onset. In FIG. 8C, synchronization is provided by an internal shared clock utilized by the sensors.

Accurate baseline blood pressure measurements can be determined from devices such as a blood pressure cuff. A baseline patient group can be measured and used to determine the relationship with measured PTT values for the group. For a large patient group, these accurate baseline blood pressure measurements, along with calculated PPT times for each of these patients, can be used to train a neural network to generate a blood pressure assessment. Alternatively, function coefficients using regression techniques can be used to determine a blood pressure assessment using PTT. However, differences in patient attributes can cause variances in the blood pressure assessment. These attributes can include, but are not limited to the parameters, gender, weight, body mass index, age, health status, blood oxygen level, heart rate, room temperature, patient temperature, and height. Health status can include factors including diseases, excessive drinking, smoking, and having or not having diabetes. The accuracy can be improved by training the neural network and utilizing these patent attributes as training inputs. A person having ordinary skill in the art of training neural networks would be able to choose the number and distribution of patients required for training the neural network.

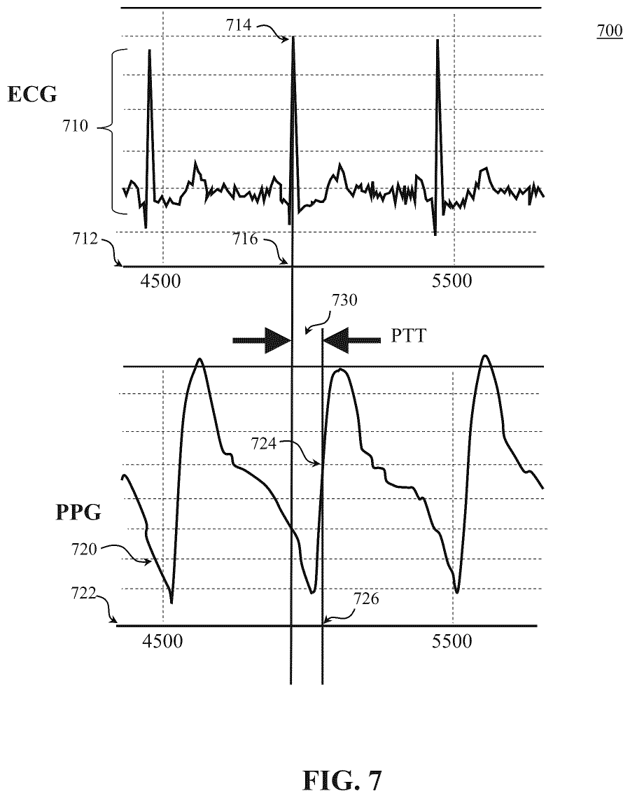

Referring to FIG. 7, graphs of two signals 700 are shown. The ECG graph 710 is a signal that can be taken from sensors located over a patient's chest or from contact points on one arm and another contact point on the opposing arm. These contact points can include the wrist or finger or points along the arm. The ECG waveform 710 is plotted along axis 7412. The cardiac onset 714 is shown as the peak of the R wave within the “qRs” complex. The “qRs” complex represents phases of a heart's electrical activity and is well-defined in the medical literature. The cardiac onset 714 is shown to occur at the corresponding time 716 crossing the time axis 712. Different points within the “qRs” complex can be used to represent the cardiac onset. These include but are not limited to the rising edge of the “R” phase, the peak of the “R” phase, the falling edge of the “R” phase.

While graph 710 shows an ECG signal, other signals and sensors can be used to detect cardiac onset. These include, but are not limited to, measuring impedance changes, detecting acoustical signals, and generating ballistocardiograph signals. Each of these signals has a distinguishing feature indicating cardiac output.

PPG graph 720 shows a signal that can be taken from sensors located over a patient's arm, preferably on their wrist and over their radial artery. The optical signal, as described above, varies with the arrival of each pulse of blood from the heart. As shown, the two graphs are synchronized in time. The PPG waveform 720 is plotted along an axis 722. The rise in the PPG signal 724 indicates the blood-arrival time into a proximal part of the limb. The cardiac onset 714 is shown as the corresponding time 726 crossing the time axis 722.

The time difference between the peak blood-arrival time 724 and the cardiac onset time 716 is the Pulse Transit Time (PTT) 730. This time is calculated by subtracting the cardiac onset time 716 from the peak blood-arrival time 726.

While graph 720 shows a PPG signal, other signals and sensors can be used to detect blood arrival. These include but are not limited to tonometry methods and pressure/force measuring methods. Each of these signals has a distinguishing feature indicating blood flow under the sensor, preferably through the radial artery under the sensor.

Referring now to FIG. 8A, an example system 800A for blood pressure assessment using a calculated PTT and patient parameters is shown. This system 800A is similar to the system of FIG. 1, but the system 800A includes additional components and features. The system 800A comprises a wrist device 810a. The wrist device 810a can include sensors 820 for generating a blood-arrival signal, patient temperature, and room temperature. Other patient parameters, such as pulse rate and blood oxygen level, can be derived from the blood-arrival sensor data.

The wrist device 810a can include electronic components for processing cardiac signal data and blood-arrival signal data to generate a PPT. The PTT and patient parameters are converted into a blood pressure assessment using a trained neural network or a preconfigured function. The preconfigured function can have its coefficients determined through iteration of data from a sample patient database. Other patient parameters can be dynamically generated and fed into the neural network, including a patient's pulse rate and blood oxygen level. Further, the wrist device 810 can include wired or wireless communication electronics for communication with a cardiac device 840 and a synchronization device 850. The wrist device 810a is worn by a patient 130 for an extended period of time and can be in the form of a watch, a bracelet, a wristband, and the like.

However, the present disclosure can include embodiments where the placement of the wrist device 810a is located at other places on a patient's body including a patient's leg.

The system 800A includes a cardiac device 840 for the generation of cardiac signal data related to cardiac activity. This generation can be through electrical signals, acoustic signals, or motion signals, using an electrical sensor, an acoustical sensor, an echocardiographic sensor, or a ballistocardiograph sensor 842. To best detect electrical, acoustical, or physical motions, the cardiac device is preferably positioned on the patient's chest over the heart.

The cardiac device 840 also includes synchronization electronics 846. The synchronization electronics 846 can include a wireless receiver for receiving a synchronization signal 855 from a synchronization device 850. This synchronization signal 855 is used by the cardiac device 840 to set an internal clock component 846 to enable the synchronized transmission of cardiac signal data. The synchronization can include digital time tags sent with the cardiac signal data and transmitted to wrist device 810a for processing. The timing of the synchronized cardiac signal data needs to be aligned with the wrist device clock 830a to within one millisecond or less.

The cardiac device 840 can include two or more electrical contacts 842A and 842B to detect electrical cardiac activity for generating cardiac signal data (ECG). These electrical contacts 842A and 842B should be in contact with the patient's skin. An alternative to an electrical or ECG sensor, an acoustic, echocardiographic, or ballistocardiograph sensor 843 can be used to generate related cardiac signal data with sufficient fidelity to determine a cardiac onset time.

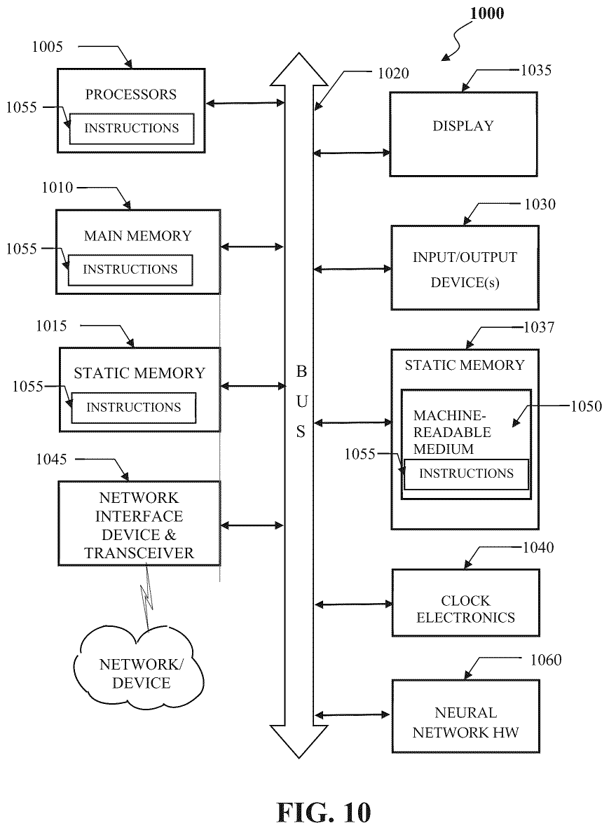

The cardiac device 840 can include a transmitter 1045 (FIG. 10) to transmit the cardiac sensor signal data by wire 844A or wirelessly 844B to the wrist device 810a. The cardiac data is used to calculate a PTT value 730 and utilized in generating a blood-pressure assessment.

The synchronization device 850 can be a global device or a low-powered local device that is located within the patient's room, home, or on the floor of a building. Global devices that can be used for synchronization include, but are not limited to, GPS (Global Positioning Service) and cellular services. A local synchronization device 850 can output a starting signal that would enable the cardiac device 840 and the wrist device 810a to start processing a cardiac signal and blood-arrival signal with a variance of less than one millisecond. Alternatively, the synchronization device 850 can send a timing message to be used by the cardiac device 840 and the wrist device 810a to time tag the data so that the data from the cardiac device 840 and the wrist device 810a can be aligned such that a PTT 730 can be accurately determined.

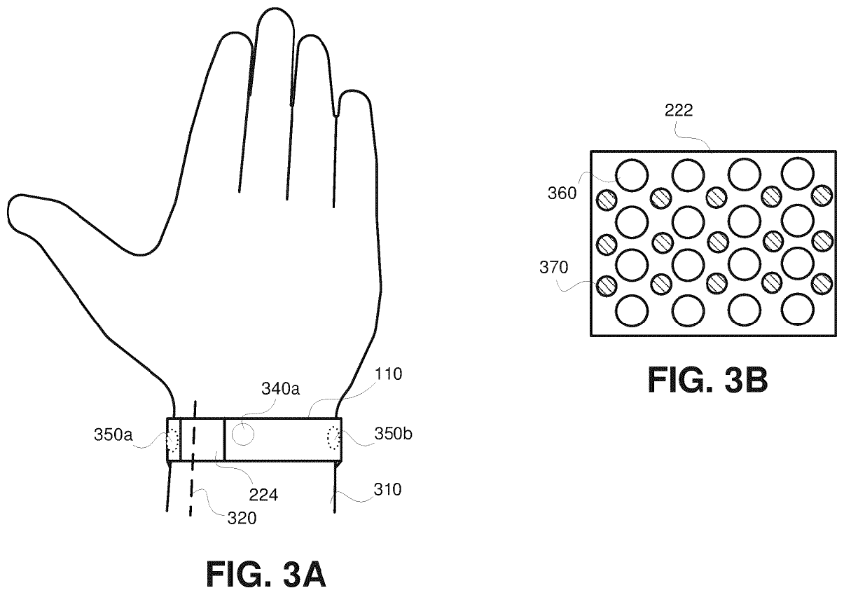

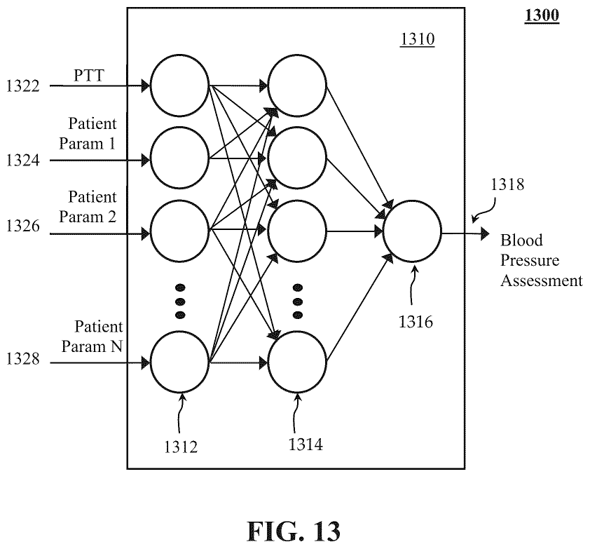

The wrist device 810a can be operable to constantly receive cardiac sensor data and generate blood-arrival sensor data via sensor 820. Based on the sensor data, wrist device 810a can be operable to generate a blood pressure assessment associated with a patient 130. Preferably, the blood-arrival sensor 820 is positioned over the wrist radial artery 320. The wrist device 810a has several similarities with the wearable device 110 described above in FIG. 3A. One difference with the wearable device 110 (FIG. 3A) is that the wrist device 810a does not include the electrical sensor 224 (FIG. 3A) and differential amplifier operable to measure the electrical signal from the wrist 310. This cardiac signal data is received through a wired or wireless receiver. The wrist device 810a can include different and additional sensors than described in FIG. 3A. Besides optical sensors, other sensors can be used to generate a blood-arrival signal, including but not limited to a tonometry sensor or a pressure-sensing sensor. Additionally, the sensors 820 can include patient temperature sensors and room temperature sensors. The wrist device includes a clock component 830a for receiving a synchronization signal 855. This clock component 830a is used to synchronize the blood-arrival signal data from sensor 820 and the received cardiac signal data from the cardiac device 840. The synchronization between the blood-arrival signal data and the cardiac signal data should be within at least one millisecond to generate an accurate PTT measurement 730 (FIG. 7) and an accurate blood pressure assessment 1318 (FIG. 13).

Optionally, the system 800A can utilize a blood pressure cuff 805 to obtain a baseline blood pressure measurement. This baseline blood pressure cuff measurement can be used, in an optional step, to provide an initial retraining of the trained neural net 1310 (FIG. 13) for the first blood pressure assessment. Subsequent blood pressure assessments can utilize the retrained neural network to provide an improved assessment.

Referring to FIG. 8B, another embodiment of a system 800B for blood pressure assessment is shown using a calculated PTT 730 (FIG. 7) and patient parameters 1-N processed by a neural network 1300 (FIG. 13) or a predetermined function. This system 800B provides the same blood pressure assessment but differs by not having the separate synchronization device 850, as depicted in FIG. 8A. The synchronization electronics is located in the clock component 830b of the wrist device 810b. The clock component 830b will send a synchronization signal to the cardiac device 840. A clock from the clock component 830b will be used internally to generate a time reference for the blood-arrival signal data. As described above, the cardiac device clock component 846 will use the synchronization signal to send synchronized cardiac signal data to the wrist device 810b. The rest of the operation of the cardiac device 840 and wrist device 810b is the same as described above.

Referring to FIG. 8C, another embodiment of a system 800C for blood-pressure assessment, is shown. This system 800C provides the same blood pressure assessment but differs from system 800A (FIG. 8A) by not having the synchronization device 850 (FIG. 8A) and does not have a cardiac device 840 (FIG. 8A). The cardiac signal is generated from one or more electrical sensors on the wrist device 810c. The synchronization electronics are part of the clock component 830c. A clock generated by the clock component 830c will be used internally to generate a time reference or clock for the data collection of the cardiac signal data and the blood-arrival signal data. The rest of the operation of the cardiac device 840 and wrist device 810c is the same as described above.

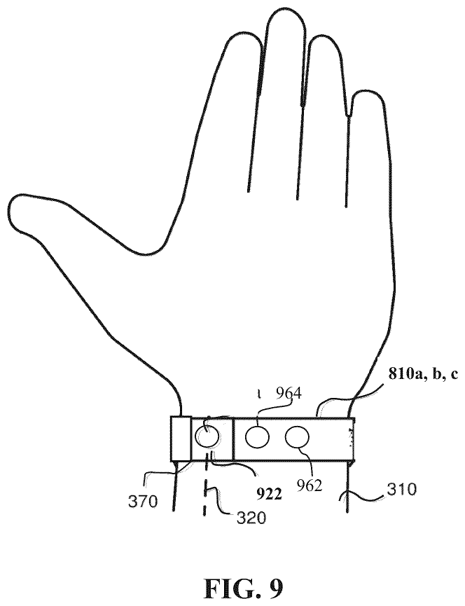

Referring to FIG. 9, a schematic diagram illustrating an example wrist device 910a, b, c placed around the left wrist 310 of a patient. This example is similar to FIGS. 3A and 3B described above.

In some embodiments, the optical sensor 920 can overlie a pulsating artery traveling along the arm and into the wrist 310. In some embodiments, the radial artery 320 passing in the inner wrist is used for measurements by the optical sensor 920. In other embodiments, other arteries, such as the ulnar artery, may be used. An external light source generating constant lighting can be used to radiate the pulsating artery. A beam reflected from the pulsating artery can be intercepted by the optical sensor 920. In certain embodiments, red lighting is used to radiate the pulsating artery. Alternatively, in other embodiments, other lighting (for example white light) can be used.

FIG. 10 is a diagrammatic representation of an example machine in the form of an electronics processing structure 1000, within which a set of instructions for causing the machine to perform any one or more of the methodologies discussed herein may be executed. All or part of this processing structure 1000 can be found in the wrist device 810a, 810b, 810c, and the cardiac device 840. The machine may be, for example, implemented in discrete components or within an integrated circuit. Further, while only a single machine is illustrated, the term “machine” shall also be taken to include any collection of machines that individually or jointly execute a set (or multiple sets) of instructions to perform any one or more of the methodologies discussed herein.

The example computer system 1000 includes a processor or multiple processors 1005 (e.g., a central processing unit (CPU), a graphics processing unit (GPU), Digital Signal Processor, Neural Processor Unit (NPU), or any combination thereof), and main memory 1010 and static memory 1015, which communicate with each other via a bus 1020. The computer system 1000 may further include a display 1035 (e.g., a liquid crystal display (LCD)) that may be on the wearable wrist device or cardiac device. The computer system 1000 may also include an alpha-numeric input device(s) 1030 (e.g., a keyboard, LEDs, analog-to-digital conversion of biometric signals including electrical cardiac signals and optical photoplethysmogram signals), a drive unit 1037 (also referred to as disk drive unit or solid-state storage), a clock device 1040, and a network interface and transceiver device 1045. The clock device 1040 maintains an accurate high-resolution clock (between 1 ms and 10 us) that is either the master clock shared with other system devices or set from an external device. The network interface device 1045 can include low-power peer-to-peer wired or wireless communications between the wrist device 810(a-c), the cardiac device 840, and the synchronization device 850. The computer system 1000 may further include a data encryption module (not shown) to encrypt data.

The drive unit 1037 includes a computer or machine-readable medium 1050 on which is stored one or more sets of instructions and data structures (e.g., instructions 1055) embodying or utilizing any one or more of the methodologies or functions described herein. The instructions 1055 may also reside, completely or at least partially, within the main memory 1010 and/or within static memory 1015 and/or within the processor(s) 1005 during execution thereof by the computer system 1000. The main memory 1010, static memory 1015, and the processors 1005 may also constitute machine-readable media.

The instructions, control, and configuration 1055 may further be transmitted or received over a network via the network interface device 1045 utilizing any one of a number of well-known transfer protocols (e.g., Hyper Text Transfer Protocol (HTTP)). While the machine-readable medium 1050 is shown in an example embodiment to be a single medium, the term “computer-readable medium” should be taken to include a single medium or multiple media (e.g., a centralized or distributed database and/or associated caches and servers) that store the one or more sets of instructions. The term “computer-readable medium” shall also be taken to include any medium that is capable of storing, encoding, or carrying a set of instructions for execution by the machine and that causes the machine to perform any one or more of the methodologies of the present application, or that is capable of storing, encoding, or carrying data structures utilized by or associated with such a set of instructions. The term “computer-readable medium” shall accordingly be taken to include, but not be limited to, solid-state memories, optical and magnetic media, and carrier wave signals. Such media may also include, without limitation, hard disks, floppy disks, flash memory cards, digital video disks, random access memory (RAM), read-only memory (ROM), and the like. The example embodiments described herein may be implemented in an operating environment comprising software installed on a computer, in hardware, or in a combination of software and hardware.

Not all components of the processing/computer system 1000 are required, and thus, portions of the computer system 1000 can be removed if not needed, such as Input/Output (I/O) devices (e.g., input device(s) 1030). One skilled in the art will recognize that the Internet service may be configured to provide Internet access to one or more computing devices that are coupled to the Internet service and that the computing devices may include one or more processors, buses, memory devices, display devices, input/output devices, and the like. Furthermore, those skilled in the art may appreciate that the Internet service may be coupled to one or more databases, repositories, servers, and the like, which may be utilized in order to implement any of the embodiments of the disclosure as described herein.

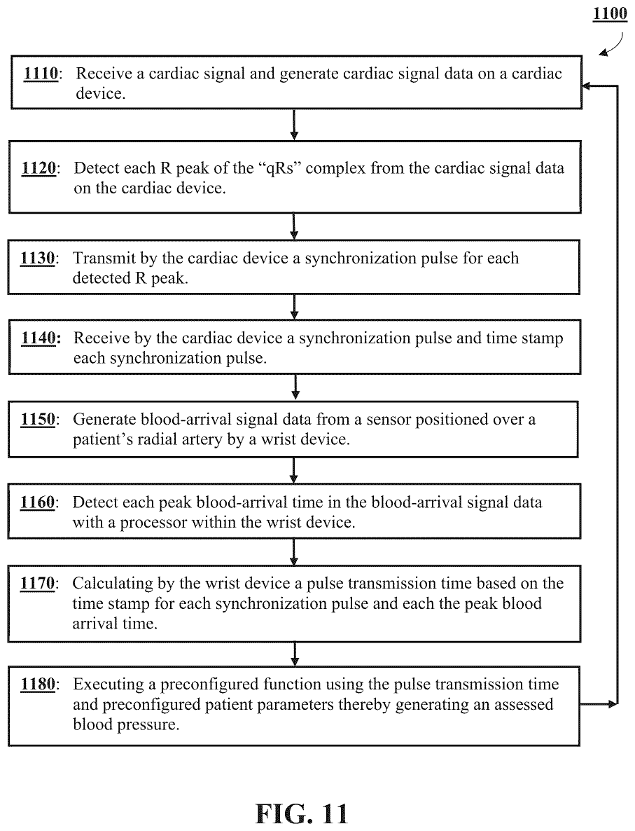

FIG. 11 is a flow chart of method 1100 for continuous blood pressure assessment using a wearable device and a cardiac device according to an example embodiment. Synchronization of each cardiac onset and the peak blood-flow signal data on the wrist device is provided through the generation of a synchronization pulse upon the detection of cardiac onset by the cardiac device. Cardiac onset is indicated by the rising edge or peak of the “R” phase of the “qRs” complex.

The synchronization pulse, which indicates the detection of cardiac onset, can be a wired or wireless pulse transmitted by the cardiac device to the wrist device. The synchronization pulse is generated with low delay or a deterministic delay. Preferably, the delay is less than two-hundred microseconds.

The synchronization pulse is symbolic and can be represented by other signals having other shapes and modulations. For a wireless synchronization pulse, a person skilled in the art of transceiver design would be able to select a wireless frequency, modulation, power level, and pulse shape that would not interfere with other equipment, could reliably be received by the wrist device, be cost-effective to implement, require low power usage, and could be integrated into the cardiac device without requiring a large footprint.

An important aspect of the synchronization pulse is that the time between the detection of the cardiac onset (R peak) and the transmission of the synchronization pulse is either short or known. A known delay can be compensated for in the PTT calculation.

In block 1110, the method 1100 a cardiac signal is received by a sensor and the cardiac signal data is generated by the cardiac device. The cardiac device is preferably placed on a patient's chest. Different cardiac sensors can be used to generate cardiac sensor data. These cardiac sensors can include, but are not limited to, ECG, acoustical, echocardiographic sensor, and ballistocardiograph sensors. For an ECG sensor, at least two electrical contacts with the patient's skin are preferable.

In block 1120, a processor or custom electronics processes the cardiac signal data to determine the “R” peak of the “qRs” complex or the rising edge of the “R” phase. This phase of an ECG signal corresponds to the cardiac onset. This step of the process can be repeated to provide continuous detection of each cardiac onset of each heartbeat.

In block 1130, upon the detection of the R peak, a synchronization pulse or signal is transmitted by the cardiac device. The transmission can be over a wireless or wired line. Preferably, the time between the cardiac onset, the detection of the cardiac onset, and the transmission of the synchronization pulse or signal is low (<200 us) or has a known and substantially invariant delay. This step of the process can be repeated for each R peak detected.

In block 1140, the wrist device receives the synchronization pulse transmitted by the cardiac device. A time stamp can be generated for each synchronization pulse or signal received.

In block 1150, the method 1100 includes the wrist device generating blood-arrival signal data from a sensor positioned over a patient's radial artery. The blood-arrival sensor can include, but is not limited to, a PPG sensor, a tonometry sensor, and a pressure-sensing sensor,

In block 1160, the method 1100 includes processing the blood-arrival sensor data to detect the peak blood-arrival time. This peak blood-arrival time is the delay between the blood flow from the heart after the cardiac onset and the blood flow reaching the wrist.

In block 1170, the method 1100 includes calculating a PTT (pulse transmission time) based on the synchronization pulse time stamp and the peak blood arrival time. If there is a known delay between the detection by the cardiac device, transmission and reception of the synchronization pulse, and time tagging the synchronization pulse, then this time can be added to the PTT time.

In block 1180, the method 1100 includes executing a preconfigured function using the PTT and preconfigured patient parameters, thereby generating an assessed blood pressure. This preconfigured function can be a trained neural network, or a function with preconfigured parameters determined through regression techniques using a database of patient data.

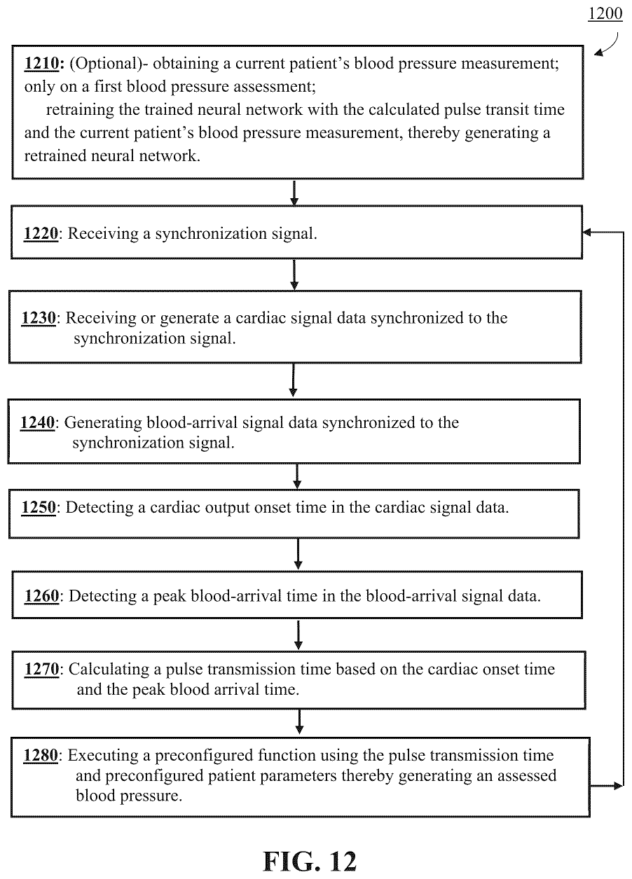

FIG. 12 is a flow chart of method 1200 for continuous blood pressure assessment using a wearable device, a cardiac device, and a synchronization device according to example embodiments. This method 1200 is also applicable to a blood pressure assessment system that does not include the cardiac device or the synchronization device but incorporates the functions of the cardiac device and synchronization function into a wrist device.

In block 1210, the method 1200 optionally can include the step of obtaining a current patient's blood pressure. A first blood pressure assessment is made measuring the PTT and using the patient's parameters. This current patient's blood pressure can accurately be measured using, for example, a blood-pressure cuff and a stethoscope in conjunction with trained medical staff. The current patient's blood pressure and the calculated PTT are used to retrain the neural network. This training can include the current patient's parameters. These parameters can include but are not limited to the patient's current temperature, pulse rate, and blood oxygen level. The retraining cycle can be repeated using the difference between the current patient's blood pressure and the blood pressure assessment to determine an error value. The error value is used to update the weights in the neural network. The retraining can continue until the error value is smaller than a predetermined error, for a fixed amount of time, or for a fixed number of training cycles.

In block 1220, the method 1200 includes one or more devices receiving a synchronization signal. These devices can include the cardiac and wrist device and any other device that needs to generate time-synchronized data. The synchronization signal can be from an external global source or a local source or is located in one of the system devices, including but not limited to a cardiac device or a wrist device. The external global source synchronization signal can be, but is not limited to, GPS (Global Positioning System) or a mobile network signal. Alternatively, the synchronization device can be near the patient, within the same room or building, and broadcast a wireless synchronization signal.

The synchronization signal is used to set an accurate clock within the cardiac and wrist devices. The synchronized devices can then generate synchronized cardiac signal data and blood flow signal data, which is used to determine an accurate PPT. The accuracy of synchronization between devices is preferably under one millisecond.

In block 1230, cardiac signal data is received or generated. The signal can be generated from an electrocardiogram (ECG) sensor, an impedance sensor, an acoustic sensor, and a ballistocardiograph sensor. In the embodiment where the cardiac device is separate from the wrist device, this data is received by a wrist device by either a wired or a wireless means. Preferably, a low-power transmission means is used. Synchronization can be provided using a digital time tag associated with one or more data elements or samples indicating the time the data was taken. For sampled digital data, the time between each sample can be constant and known by the receiving device. Alternatively, the time between samples can be included as part of a digital time tag or received as part of an initialization process.

In another embodiment, the cardiac signal is measured by the wrist device through contact with the wrist and a finger or other part of the opposing hand. The synchronization signal can be a clock on the wrist device and used to clock the electronics that generate ECG signal data. The ECG signal data can be digital data samples. This ECG sampled signal data can be time-stamped to correlate with the blood-arrival signal data. The same clock can be used to generate the blood-arrival signal data.

In block 1240, a blood-arrival signal is generated. The blood-arrival signal is synchronized with the synchronization signal. In one embodiment, the synchronization signal, or master clock, is internal to one of the devices, preferably the wrist device. The master clock can be used to sample a signal associated with the blood-arrival signal. As discussed and disclosed above, this signal can be from an optical sensor used for generating a PPG signal that corresponds with the blood-arrival signal. Other sensors can be used that generate a signal that corresponds to the blood-arrival signal, including but not limited to a tonometry sensor or a pressure sensor.

In block 1250, the cardiac signal data is processed to determine the synchronized time when the cardiac output occurred. The cardiac output occurs on the rising edge or peak of the “R” phase of the “qRs” complex. This rising edge is shown in FIG. 7 at 714. However, the “R” is applicable only to sensor that monitor the electrical activity of the heart. If other types of sensor, monitoring different physical attributes, then a different signal characteristic can be used to identify the cardiac onset. For example, for an acoustic sensor, a specific sound with a specific acoustic signature can signify the cardiac onset time.

In block 1260, the blood-arrival signal data is processed to determine when the synchronized peak blood-arrival time occurs. The peak blood-arrival occurs on the rising edge of the blood-arrival signal. This rising edge is shown in FIG. 7 at 724.

In block 1270, the PTT (pulse transit time) is calculated by subtracting the peak blood-arrival time 726 from the cardiac onset time 716. This time is shown in FIG. 7 as the PPT 730.

In block 1280, a blood-pressure assessment is determined using the calculated PTT and one or more patient parameters. The patient parameters can include gender, height, weight, BMI (body mass index), age, health status, blood oxygen level, heart rate, room temperature, and patient temperature.

The PTT and patient parameters are input into a trained neural network or into a function with preset coefficients. The output of the trained neural network or function is a blood pressure assessment, which should be close to the patient's blood pressure. Patient parameters that do not quickly change, like gender, weight, BMI, age, and health status, can be entered into the wrist device through a user interface or transmitted to the wrist device. Since the wearable device can include sensors for detecting the patient's current pulse rate, temperature, blood oxygen level, and room temperature, these parameters can be inputted once or updated for each blood pressure assessment cycle.

The pulse rate can be determined from the previously described cardiac sensor signal using the time between the “qRs” complex peaks or from the blood-arrival signal data. The blood oxygen level can be determined from the optical sensors as described above. The patient's temperature and the patient's room temperature can be determined from sensors on the wrist device. The blood pressure assessment method can be repeated for a continuous assessment by jumping back to step 1220 upon the completion of this step.

Referring to FIG. 13, a trained neural network 1300 is illustrated. The trained neural network 1310 shown is a Radial Basis Neural Network, which is well suited for function approximation. However, other neural network architectures are contemplated. This trained neural network can be implemented in software or in a hardware implementation of a neural network processor, as shown in element 1360 (FIG. 13). The trained neural network 1310 is comprised of the input layer 1310, the hidden layer 1314, and the output node 1316. Only one hidden layer 1314 is shown, but more hidden layers can be included in the neural network model.

The inputs to the trained neural network 1310 include a calculated PTT 1322 and patient parameters 1324, 1326, and 1328. The patient parameters can include one or more of the parameters, gender, weight, body mass index, age, health status, blood oxygen level, heart rate, room temperature, patient temperature, and height. The output of the neural network 1318 is the sum of the last hidden layer representing a blood pressure assessment.

Training the neural network 1310 with patient data is required to generate accurate blood pressure assessments. A patient information database can be used that includes a statistically relevant distribution of patient parameters. This may require hundreds of patients to provide a distribution over all the parameters. This information database includes measurements of the patient's baseline blood pressure, calculated PPTs for each patient, and a patient's parameters. Each of the patient's information is input into the neural network 1310. The difference between the blood pressure assessment 1318 and the patient's baseline blood pressure represents the error that is used as feedback to train the neural network hidden layers 1314. A person skilled in the art of training neural networks would know the training parameters required to train the neural network and any required data normalization.

Some of the patient parameters are static, and some change over short periods of time. Parameters like age or weight will not change over a short period of time. However, patient's parameters, such as pulse rate and blood oxygen level, could change in the span of a minute. Thus, in one embodiment, these parameters can be updated each time a blood pressure assessment is calculated. The other static parameters are not updated between blood pressure assessments.

In another embodiment, the trained neural network 1310 is retrained when first used on a patient. This retraining of the neural network is described above in step 1310. A patient's blood pressure can be taken using a direct and accurate means, for example, using a blood pressure cuff. The trained neural network 1300 is then retrained with this patient's current blood pressure and a current PTT. Thus, any bias or error in the trained neural network may be reduced.

### ECG Wearable Device

The present disclosure provides systems and methods for measuring ECG data using a wearable device. Embodiments of the present disclosure can allow measuring ECG data of a patient in a non-intrusive manner while, for example, the patient is at home, at work, outdoors, traveling, or is located in some other stationary or mobile environment. Some embodiments of the present disclosure include the wearable device that the patient wears around a wrist. The wearable device allows measuring ECG data from the patient's wrist without requiring the patient to take an active role in the process. The ECG data collected during an extended period of time can be analyzed to detect and track trends and to make conclusions concerning symptoms and a progression of one or more chronic diseases from which the patient might suffer.

According to some example embodiments, a method for measuring ECG data using a wearable device includes recording an electrical signal from a patient's wrist. The electrical signal can be recorded via at least one electrical sensor associated with the wearable device. The electrical signal can include an ECG signal and a noise. The method allows splitting the electrical signal into segments. The splitting can be based on a reference signal. The reference signal can include an indication of onset times of heart beats. The method can include averaging the segments to derive average ECG data.

Referring now to FIG. 1, an example system 100 for measuring ECG data using a wearable device is shown. The system 100 includes at least the wearable device 110. The wearable device can include sensors 120. In some embodiments, the wearable device 110 is worn by a patient 130 (for example, on a wrist) for an extended period of time. The wearable device 110 can be carried out as a watch, a bracelet, a wristband, and the like.

The wearable device 110 can be operable to constantly collect, via sensors 120, sensor data from a patient 130. Based on the sensor data, the wearable device 110 can be operable to obtain ECG data associated with the patient 130.

In some embodiments, the system 100 includes a mobile device 140. The mobile device 140 can be communicatively coupled to the wearable device 110. In various embodiments, the mobile device 140 is operable to communicate with the wearable device 110 via a wireless connection, including but not limited to Wi-Fi, Bluetooth, and Infrared (IR). The mobile device 140 can include a mobile phone, a smart phone, a phablet, a tablet computer, a notebook, and so forth. The mobile device 140 can be operable to receive the sensor data and analyze the sensor data to generate ECG data.

In further embodiments, the system 100 may include a cloud-based computing resource 150 (also referred to as a computing cloud). In some embodiments, the cloud-based computing resource 150 includes one or more server farms/clusters comprising a collection of computer servers and is co-located with network switches and/or routers. In certain embodiments, the mobile device 140 is communicatively coupled to the computing cloud 150. The mobile device 140 can be operable to send the sensor data to the computing cloud 150 for further analysis (for example, for extracting ECG data from the sensor data and storing the results). The computing cloud 150 can be operable to run one or more applications and to provide reports regarding health status of the patient based on trends in ECG data over time. A doctor 170 treating the patient 130 may access the reports (for example, via computing device 160) using the Internet or a secure network. In some embodiments, the results of the analysis of the medical parameters can be sent back to the mobile device 140.

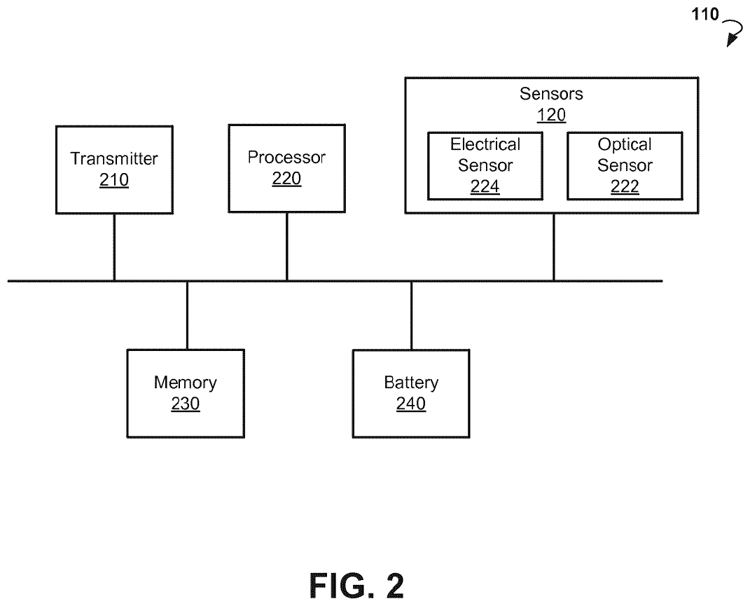

FIG. 2 is a block diagram illustrating components of wearable device 110, according to an example embodiment. The example wearable device 110 includes sensors 120, a transmitter 210, a processor 220, memory 230, and a battery 240. The wearable device 110 may comprise additional or different components to provide a particular operation or functionality. Similarly, in other embodiments, the wearable device 110 includes fewer components that perform similar or equivalent functions to those depicted in FIG. 2.

The transmitter 210 can be configured to communicate with a network such as the Internet, a Wide Area Network (WAN), a Local Area Network (LAN), a cellular network, and so forth, to send data streams (for example, sensor data, ECG data, and messages).

The processor 220 can include hardware and/or software, which is operable to execute computer programs stored in memory 230. The processor 220 can use floating point operations, complex operations, and other operations, including processing and analyzing sensor data, to extract ECG data.

In some embodiments, the battery 240 is operable to provide electrical power for the operation of other components of the wearable device 110. In some embodiments, the battery 240 is a rechargeable battery. In certain embodiments, the battery 240 is recharged using an inductive charging technology.

In various embodiments, the sensors 120 include at least one electrical sensor 224 and at least one optical sensor 222. In certain embodiments, the sensor 120 can include position and motion sensors. The motion sensors can include an accelerometer, gyroscope, and Inertial Measurement Unit (IMU).

FIG. 3A is a schematic diagram illustrating an example wearable device 110 placed around the left wrist 310 of a patient. In the example of FIG. 3A, the wearable device 110 is carried in a shape of a watch, a ring, and/or a bracelet.

The electrical sensor 224 can include a differential amplifier operable to measure the electrical signal from the wrist 310. The electrical sensor 224 can include two active amplifier input plates embedded in the wearable device at opposite ends. In some embodiments, the first input plate (not shown) can be placed above the outer side of the wrist, and the second input plate 340a can be placed beneath the inner side of the wrist 310. Alternatively, or additionally, in other embodiments, the input plates 350a and 350b can be placed in contact with, respectively, the left and right sides of the wrist 310.

In some embodiments, the optical sensor 222 can be placed beneath a pulsating artery traveling along the arm and into the wrist 310. In some embodiments, the radial artery 320 passing in the inner wrist is used for measurements by the optical sensor 222. In other embodiments, other arteries, such as the ulnar artery, may be used. An external light source generating constant lighting can be used to radiate the pulsating artery. A beam reflected form the pulsating artery can be intercepted by the optical sensor 222. In certain embodiments, red lighting is used to radiate the pulsating artery. Alternatively, in other embodiments, other lighting, for example white lighting, can be used.

FIG. 3B is a schematic diagram showing an optical sensor 222, according to an example embodiment. The optical sensor 222 can include multiple light sensors 360 (for example, photoelectric cells) to measure the reflected light. The pulsating artery can be irradiated by multiple light transmitters 370 (for example, Light Emission Diodes (LEDs)). The number and location of the light sensors and light transmitters can be chosen in a way that if, accidentally, the wearable device 110 slides off, at least one of the light sensors is still located sufficiently close to the pulsating artery. In some embodiments, when measuring the light reflected from the pulsating artery, a signal from those photoelectric cells that provides the strongest output can be selected for further processing.

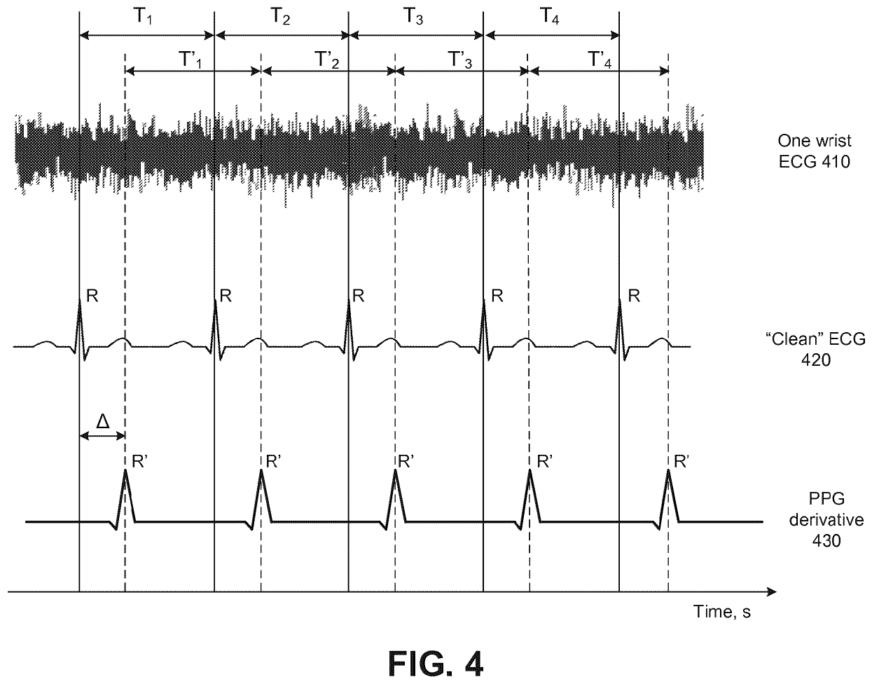

FIG. 4 shows plots of an example electrical signal 410 measured from one wrist (or some other limb), an example plot of “clean” ECG signal 420, and a plot of the first derivative 430 of a PPG signal. The electrical signal can be recorded with electrical sensor 224 using input plates placed on the wearable device 110. Taking measurements from a single hand or a single wrist can be challenging because the difference in voltages between measured locations is miniscule. The electrical signal 410 measured at the wrist can include an ECG signal and a noise. The noise can be caused by muscle activity, patient movements, and so forth. The noise component can be larger than the ECG signal. In some embodiments, the signal-to-noise ratio (SNR) is in the range of −40 dB to −60 dB.

The “clean” ECG signal 420 is an imaginary ECG signal that can be obtained simultaneously with electrical signal 410 using a regular two leads ECG recording, for example, when two input plates of a cardiograph are placed at two different wrists of the patient. The “clean” ECG signal 420 can include R peaks corresponding to heart beats. Using the “clean” ECG signal 420 as a reference, the electrical signal 410 can be split in segments, with each of the segments Ti (i=1, 2, 3 . . . ) corresponding to one RR interval (an interval between two successive heart beats). Each segment Ti (i=1, 2, 3 . . . ) of an electrical signal can contain an ECG signal si(t) and a noise component ei(t). Assuming a stationary heartbeat waveform, if segments Ti (i=1, 2, 3 . . . ) are substantially of the same length, then signal si(t) is substantially of the same waveform when noise components ei(t) are not correlated to each other. The following averaging technique can be used to extract an ECG signal S(t) from electrical signal 410:

S(t)=Σᵢ₌₁^{p}(sᵢ(t)+eᵢ(t)),

wherein p is a number of segments Ti (i=1, 2, 3 . . . ) selected for averaging. The segments Ti (i=1, 2, 3 . . . ) selected for averaging are measured for a pre-determined time period (for example, for a few seconds or a few minutes). The segments selected for averaging are substantially of the same length.

The averaging increases the SNR in resulting average ECG signal S(t). In certain embodiments, the SNR in resulted averaged ECG signal S(t) can be further increased by weighted averaging, Wiener filtering, Adaptive filtering, and with other techniques.

Since the “clean” ECG signal 420 is not available when the measurements are carried out on a single wrist, a PPG signal can be used as a reference signal to split the electrical signal 410 into segments. In various embodiments of the present disclosure, the PPG signal is recorded using the optical sensor 222 simultaneously with the electrical signal 410, which is recorded by the electrical sensor 224. The PPG signal is obtained by sensing a change in the color of skin. The change of skin color is caused by a blood flow in the pulsating artery. In some embodiments, the first derivative 430 of PPG can be used as a reference signal. The first derivative 430 of PPG signal can include sharp peaks R′ corresponding to the heart beats. Since it takes a time for blood to flow from the heart to the wrists, the peaks R′ are shifted by a time period Δ relative to the heart beats R in “clean” ECG signal 420. Assuming that A is approximately the same for all heart beats, the peaks R′ can be used to split the electrical signal 410 in segments T′I (i=1, 2, 3 . . . ). In some embodiments, the averaging technique can be applied to segments T′i (i=1, 2, 3 . . . ) of ECG data to increase SNR. In other embodiments, the segments T′i (i=1, 2, 3 . . . ) can be shifted by the time period Δ and the averaging can be applied to the shifted segments.

In some embodiments, prior to the averaging, the segments of the ECG signal can be split into groups. Each of these groups can include segments of the ECG signal corresponding to a certain pulse rate at which the segments were measured. The pulse rate is provided based on measurements of the PPG signal. The averaging can be applied to each group of segments independently.

In some embodiments, averaging can be performed on ECG data collected within a pre-determined period of time (for example, during a day). The average ECG data that is obtained by averaging segments collected during a day can be further compiled and saved locally (in the memory of wearable device 110 or mobile device 140) or remotely (in a memory storage of computing cloud 150) for further analysis. The average data can be analyzed to detect and track changes and trends in average ECG data over an extended period of time. The extended period of time can include one or more weeks, one or more months, or even years.

In some embodiments, based at least on the trends, symptoms of one or more chronic diseases are indicated due to their relationship with measured or derived parameters. In certain embodiments, reports concerning suspected progression of one or more chronic diseases can be generated based on the trends. In some embodiments, based on the symptoms, the patient can be advised to take palliative steps such as taking a medication and/or to contact a medical professional.

In various embodiments, processing electrical signal 410 and first derivative 430 of PPG signal, analyzing average ECG data to detect and track trends, and generating reports on symptoms and progression of chronic diseases can be performed locally on wearable device 110 and/or mobile device 140 and remotely in computing cloud 150.

It may be desirable to utilize the motion data obtained via the motion sensors to provide parameters of body movement and tremor. In certain embodiments the motion data can be used for performing a noise analysis to remove artifacts in ECG data.

In further embodiments, an accelerometer (a tri-axis accelerometer) can be placed on skin near an artery of the patient and provide data on flexion of the artery due to blood flow. The data provided by the accelerometer can be used to generate the ECG data.

The wearable device 110 can be configured to operate in at least two modes. A first mode can include reading an ECG data from one wrist, synchronizing the ECG data with reference PPG data, segmenting and grouping the ECG data to perform averaging ECG data to improve the SNR.

In a second mode, the wearable device can be configured to improve the PPG data based on ECG data as a reference signal. While operating the wearable device in the second mode, the patient can be asked to touch the wearable device with other hand, in order to allow receiving a “two-handed” ECG data (good quality ECG data) which include much less noise than “a single wrist” ECG data. The wearable device can include an extra lead to receive input from the other hand when touching. In the second mode, poor PPG data can be segmented using the “two-handed” ECG data as a reference signal. The PPG segments can be dropped and averaged to receive high quality PPG data. The high quality PPG data can be used along with good quality ECG data to estimate, for example, a pulse travel time.

In some embodiments, the system 100 for measuring ECG data includes at least one additional wearable device. The additional wearable device can be identical to the wearable device 110. In some embodiments, patient 130 may wear one of the devices throughout the day and another device at nighttime. In certain embodiments, the wearable device can be changed when the battery level drops below a certain level. The wearable device that is not in use at that moment can be recharged. In some further embodiments, the device can be recharged using induction charging technology. In some embodiments, since both the devices are in communication with mobile device 140, the replaced device can, at least partially, transmit recorded information (ECG data and PPG data) to the replacing wearable device for synchronization. The information can be downloaded to the mobile device 140 and the mobile device 140 can be operable to send the information to the replacing device. Additionally in other embodiments, the two wearable devices can be configured to exchange information (for example ECG data and PPG data) via the computing cloud 150.

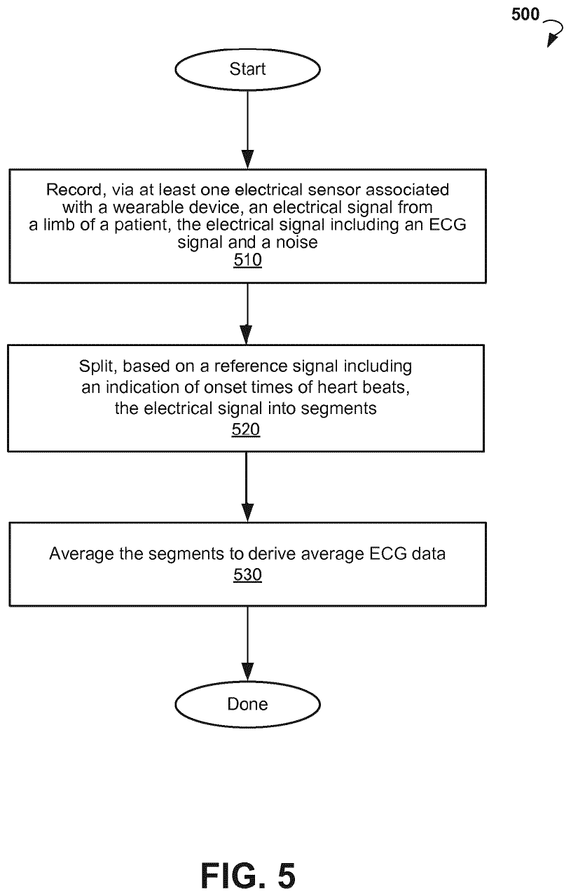

FIG. 5 is a flow chart of a method 500 for measuring ECG data from using a wearable device, according to an example embodiment. In block 510, method 500 includes recording an electrical signal from a wrist of a patient. The electrical signal can be recorded by at least one electrical sensor associated with the wearable device. The wearable device can be in a shape of a watch, a bracelet, or a wristband configured to be worn on the wrist of the patient. The electrical signal includes an ECG signal and a noise.

In block 520, the method 500 can include splitting the electrical signal into segments based on a reference signal. The reference signal can include an indication of onset times of heart beats. In some embodiments, the reference signal includes a PPG signal recorded via an optical sensor associated with the wearable device simultaneously with the electrical signal. In certain embodiments, the reference signal is the first derivative of a PPG signal recorded via the optical sensor simultaneously with the electrical signal. The optical sensor can be configured to measure color of skin beneath a pulsating artery of the wrist.

In block 530, the method 500 proceeds to average the segments and to derive average ECG data. Averaging the segments improves the SNR in the electrical signal.

### Example

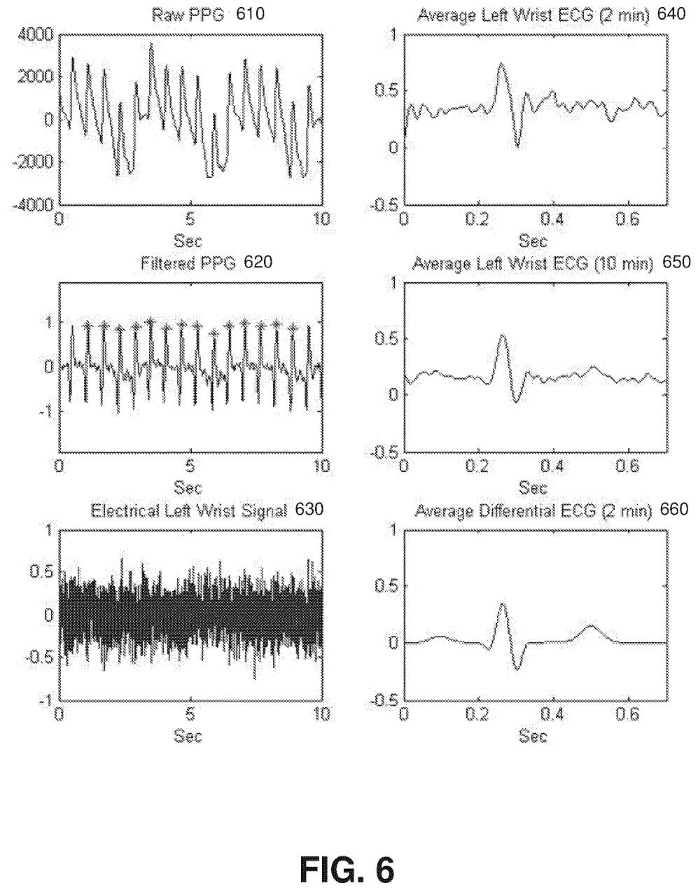

FIG. 6 illustrates plots of a raw PPG signal (optical signal) 610, a filtered PPG signal 620, an electrical signal 630, average ECG signals 640 and 650, and an average differential ECG signal 660. Recordings of the electrical signal 630 and a raw PPG signal 610 can be simultaneously measured at a wrist of a patient. The electrical signal 630 can be measured using a differential amplifier, and the optical signal can be measured with a photodiode detector placed beneath the radial artery. The raw PPG (optical) signal 610 can be processed to receive filtered PPG signal 620, yielding trigger points for the time-locked averaging of the ECG signal.

According to example measurements, an average 5-minute ECG signal and 15-minute average ECG signal are shown to converge to a two-sided average ECG signal. The two-sided average ECG signal is measured from both sides (for example, from two different wrists of patient). The ECG complex is already evident in a 5-minute average, yet a 15-minute recording provides a higher quality average waveform.

The present technology is described above with reference to example embodiments. Therefore, other variations upon the example embodiments are intended to be covered by the present disclosure.

The corresponding structures, materials, acts, and equivalents of all means or step plus function elements in the claims below are intended to include any structure, material, or act for performing the function in combination with other claimed elements as specifically claimed. The description of the present technology has been presented for purposes of illustration and description but is not intended to be exhaustive or limited to the present technology in the form disclosed. Many modifications and variations will be apparent to those of ordinary skill in the art without departing from the scope and spirit of the present technology. Exemplary embodiments were chosen and described in order to best explain the principles of the present technology and its practical application and to enable others of ordinary skill in the art to understand the present technology for various embodiments with various modifications as are suited to the particular use contemplated.

Aspects of the present technology are described above with reference to flowchart illustrations and/or block diagrams of methods, apparatus (systems), and computer program products according to embodiments of the present technology. It will be understood that each block of the flowchart illustrations and/or block diagrams and combinations of blocks in the flowchart illustrations and/or block diagrams can be implemented by computer program instructions. These computer program instructions may be provided to a processor of a general-purpose computer, special-purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer-readable medium that can direct a computer, other programmable data processing apparatus, or other devices to function in a particular manner, such that the instructions stored in the computer-readable medium produce an article of manufacture, including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus, or other devices to produce a computer-implemented process such that the instructions which execute on the computer or other programmable apparatus provide processes for implementing the functions/acts specified in the flowchart and/or block diagram block or blocks.

The flowchart and block diagrams in the Figures illustrate the architecture, functionality, and operation of possible implementations of systems, methods, and computer program products according to various embodiments of the present technology. In this regard, each block in the flowchart or block diagrams may represent a module, section, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or combinations of special purpose hardware and computer instructions.

In the following description, for purposes of explanation and not limitation, specific details are set forth, such as particular embodiments, procedures, techniques, etc., in order to provide a thorough understanding of the present invention. However, it will be apparent to one skilled in the art that the present invention may be practiced in other embodiments that depart from these specific details.

Reference throughout this specification to “one embodiment” or “an embodiment” means that a particular feature, structure, or characteristic described in connection with the embodiment is included in at least one embodiment of the present invention. Thus, the appearances of the phrases “in one embodiment” or “in an embodiment” or “according to one embodiment” (or other phrases having similar import) at various places throughout this specification are not necessarily all referring to the same embodiment. Furthermore, the particular features, structures, or characteristics may be combined in any suitable manner in one or more embodiments. Furthermore, depending on the context of discussion herein, a singular term may include its plural forms, and a plural term may include its singular form. Similarly, a hyphenated term (e.g., “on-demand”) may occasionally be interchangeably used with its non-hyphenated version (e.g., “on demand”), a capitalized entry (e.g., “Software”) may be interchangeably used with its non-capitalized version (e.g., “software”), a plural term may be indicated with or without an apostrophe (e.g., PE's or PEs), and an italicized term (e.g., “N+1”) may be interchangeably used with its non-italicized version (e.g., “N+1”). Such occasional interchangeable uses shall not be considered inconsistent with each other.

Also, some embodiments may be described in terms of “means for” performing a task or set of tasks. It will be understood that a “means for” may be expressed herein in terms of a structure, such as a processor, a memory, an I/O device such as a camera, or combinations thereof. Alternatively, the “means for” may include an algorithm that is descriptive of a function or method step, while in yet other embodiments the “means for” is expressed in terms of a mathematical formula, prose, or as a flow chart or signal diagram.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

It is noted that the terms “coupled,” “connected,” “connecting,” “electrically connected,” etc., are used interchangeably herein to generally refer to the condition of being electrically/electronically connected. Similarly, a first entity is considered to be in “communication” with a second entity (or entities) when the first entity electrically sends and/or receives (whether through wireline or wireless means) information signals (whether containing data information or non-data/control information) to the second entity regardless of the type (analog or digital) of those signals. It is further noted that various figures (including component diagrams) shown and discussed herein are for illustrative purpose only and are not drawn to scale.

If any disclosures are incorporated herein by reference and such incorporated disclosures conflict in part and/or in whole with the present disclosure, then to the extent of conflict, and/or broader disclosure, and/or a broader definition of terms, the present disclosure controls. If such incorporated disclosures conflict in part and/or in whole with one another, then to the extent of conflict, the later-dated disclosure controls.

While various embodiments have been described above, it should be understood that they have been presented by way of example only, and not limitation. The descriptions are not intended to limit the scope of the invention to the particular forms set forth herein. To the contrary, the present descriptions are intended to cover such alternatives, modifications, and equivalents as may be included within the spirit and scope of the invention as defined by the appended claims and otherwise appreciated by one of ordinary skill in the art. Thus, the breadth and scope of a preferred embodiment should not be limited by any of the above-described exemplary embodiments.

What is claimed is:

1. A method of blood pressure assessment comprising:
receiving a synchronization signal;
generating, by a cardiac device, cardiac signal data that is synchronized to the synchronization signal;
transmitting the cardiac signal data from the cardiac device to the wrist device;
generating, by a wrist device, blood-arrival signal data that is synchronized to the synchronization signal;
determining a cardiac output onset time within the cardiac signal data;
determining a peak blood-arrival time in the blood-arrival signal data;
calculating a pulse transit time based on the cardiac onset time and the peak blood-arrival time; and
generating an assessed blood pressure executing a preconfigured function using the pulse transit time and preconfigured patient's parameters thereby generating an assessed blood pressure.

2. The method of claim 1, wherein the synchronization signal is provided by synchronization electronics within the wrist device and transmitted to the cardiac device.

3. The method of claim 1, wherein the cardiac signal data are digital samples and the cardiac signal data includes one or more time-tags associated with the digital samples and synchronized with the synchronization signal.

4. The method of claim 1, wherein the cardiac signal data is generated by one of an ECG sensor, an acoustical sensor, an echocardiographic sensor, and a ballistocardiograph sensor.

5. The method of claim 1, wherein the blood-arrival signal data is generated by one of a PPG sensor, a tonometry sensor, and a pressure-sensing sensor.

6. The method of claim 1, wherein the preconfigured function is one of a trained neural network and a regression function.

7. The method of claim 6, wherein the patient's parameters include one or more of gender, weight, body mass index, age, health status, blood oxygen level, heart rate, room temperature, patient temperature, and height.

8. The method of claim 6, further comprising:
obtaining a current patient's blood pressure measurement only on a first blood pressure assessment;
retraining the trained neural network or refitting the regression function with the calculated pulse transit time and the current patient's blood pressure measurement, thereby generating a retrained neural network or a refitted regression function; and
repeating a plurality of blood pressure assessments using the retrained neural network or the refitted regression function.

9. A system for blood pressure assessment comprising:
a cardiac device comprising:
a cardiac receiver configured to receive a synchronization signal;
cardiac electronics configured to receive a cardiac signal and generate cardiac signal data synchronized to the synchronization signal;
a cardiac transmitter configured to transmit the cardiac signal data; and
a wrist device comprising:
a wrist receiver configured to input the synchronization signal and the cardiac signal data;
wrist electronics configured to receive the cardiac signal data and a wrist sensor configured to generate blood-arrival signal data that is synchronized to the synchronization signal, the wrist sensor positioned over a patient's radial artery;
a processor configured to execute instructions to:
determine a cardiac onset time in the cardiac signal data;
determine a peak blood-arrival time in the blood-arrival signal data;
calculate a pulse transit time based on the onset time and the peak blood-arrival time; and
execute a preconfigured function using the pulse transit time and preconfigured patient's parameters thereby generating an assessed blood pressure.

10. The system of claim 9, wherein the wrist device further comprises synchronization electronics providing the synchronization signal.

11. The system of claim 9, wherein the wrist device sends a synchronization time to the cardiac device.

12. The system of claim 9, wherein the cardiac signal data are digital samples and the cardiac signal data includes one or more time-tags associated with the digital samples and synchronized with the synchronization signal.

13. The system of claim 9 wherein the cardiac device includes one of an ECG sensor, an acoustical sensor, an echocardiographic sensor, and a ballistocardiograph sensor.

14. The system of claim 9, wherein the blood-arrival signal data is generated by one of a PPG sensor, a tonometry sensor, and a pressure-sensing sensor.

15. The system of claim 9, wherein the preconfigured function is one of a trained neural network and a regression function.

16. The system of claim 15, wherein the patient's parameters include one or more of gender, weight, body mass index, age, health status, blood oxygen level, heart rate, room temperature, patient temperature and height.

17. The system of claim 15, further comprising:
obtaining a current patient's blood pressure measurement only on a first blood pressure assessment;
retraining the trained neural network or refitting the regression function with the calculated pulse transit time and the current patient's blood pressure measurement, thereby generating a retrained neural network or regression function; and
repeating a plurality of blood pressure assessments using the retrained neural network or the refitted regression function.

18. A system for continuous blood pressure assessment comprising:
a cardiac device configured to be placed on a patient's chest, the cardiac device comprising:
a cardiac transmitter configured to transmit a synchronization pulse;
cardiac electronics configured to receive a cardiac signal and generate cardiac signal data;
a cardiac processor configured to execute instructions to:
determine each “R” peak of the qRs complex from the cardiac signal data; and
transmit a synchronization pulse for each determined R peak;
a wrist device comprising:
a wrist receiver configured to receive each synchronization pulse and generate a time stamp for each received synchronization pulse;
wrist electronics configured to generate blood-arrival signal data from a sensor positioned over a patient's radial artery;
a wrist processor configured to execute instructions to:
determine each peak blood-arrival time in the blood-arrival signal data;
calculate a pulse transit time for each peak blood-arrival time based on the time stamp for each of the received synchronization pulse and each determined peak blood-arrival time; and
execute a preconfigured function using the pulse transit time and preconfigured patient's parameters thereby generating an assessed blood pressure.

19. The system of claim 18, wherein the synchronization pulse is a wireless pulse with a low or known delay between the determination of the R peak and transmission of the synchronization pulse.

20. The system of claim 18, wherein the cardiac electronics to receive the cardiac signal includes one of an ECG sensor, an acoustical sensor, an echocardiographic sensor, and a ballistocardiography sensor.

21. The system of claim 18, wherein the preconfigured function is one of a trained neural network and a regression function.

22. The system of claim 21, wherein the preconfigured patient's parameters include one or more of gender, weight, body mass index, age, health status, blood oxygen level, heart rate, room temperature, patient temperature and height.

23. The system of claim 18, wherein the blood-arrival signal data is generated by one of a PPG sensor, a tonometry sensor, and a pressure-sensing sensor.
